(19) United States
(12) Patent Application Publication
(10) Pub. No.: US 2025/0032696 A1
(43) Pub. Date: Jan. 30, 2025

(54) MACHINE FOR THE PURIFICATION OF ADIPOSE TISSUE AND INTRODUCTION OF THE PURIFIED ADIPOSE TISSUE INTO A PATIENT'S BODY

(71) Applicant: NEOSYAD, AIX-EN-PROVENCE (FR)

(72) Inventors: Régis ROCHE, LORGUES (FR); François CABAUD, EGUILLES (FR); Xavier NELISSEN, VILLERS SAINT SIMEON (BE)

(21) Appl. No.: 18/717,112

(22) PCT Filed: Nov. 3, 2022
(86) PCT No.: PCT/FR2022/052073

§ 371 Date: Jun. 6, 2024

(30) Foreign Application Priority Data
Dec. 8, 2021 (FR) FR2113137

(51) Int. Cl. A61M 1/00

(52) U.S. Cl. CPC A61M 1/895; A61M 1/79; A61M 1/892; A61M 2202/08; A61M 2205/103; A61M 2205/7545

(57) ABSTRACT

Disclosed herein is a machine for the purification of adipose tissue and introduction of the purified tissue into a patient's body, the machine comprising at least: a material circulation circuit comprising at least a purification device able to purify adipose tissue removed from the patient's body, the purification device comprising means for retaining adipose tissue and filtering contaminants delimiting a treatment volume intended to receive the adipose tissue, and a rotational drive means able to rotate the retaining and filtering means, and (ii) an introduction device able to communicate with the purification device for introducing purified adipose tissue into the patient's body.

### CROSS REFERENCE TO RELATED APPLICATIONS

This application claims priority to PCT/FR2022/052073 filed on Nov. 3, 2022 which claims priority to FR2113137 filed on Dec. 8, 2021. The contents of each of which are incorporated by reference herein.

### TECHNICAL FIELD

The present application concerns a machine for the purification of adipose tissue and introduction of the purified tissue into a patient's body, as well as associated assemblies. The application especially finds application for autologous adipose tissue transplantation procedures (“lipofilling”) with aesthetic and reconstructive aims. In particular, the application finds a use for breast surgery operations; however, the implementation of the machine that is the subject of application is not limited to this application.

### PRIOR ART

Autologous adipose tissue transplantation procedures are used in surgery, especially breast surgery, to shape the breast to give it a more natural appearance after deep inferior epigastric perforator (DIEP), latissimus dorsi flap reconstruction, or after placing a breast implant. These operations involve taking fat from a patient in a donor area and then reintroducing it into the area of interest of the patient's body. These operations currently do not have a reproducible rendering and may require several interventions due to the resorption of the introduced material (loss of volume encountered after the operation). In addition, the fat is prepared for its reintroduction by an operator, surgeon or surgical assistant, which generates a relatively high cost of implementing the operation and a risk of error or loss of reproducibility. It is therefore desirable to propose a technique which simplifies and makes existing operations more reliable.

The document US 2020/0054824 sought to propose an improvement by implementing a closed loop system in which the fat is harvested to be prepared with a view to its reintroduction by being mixed with a washing liquid in a gravity filtration device marketed under the commercial reference Revolve®, and then to be reintroduced into the patient's body using a centrifugal pump. However, it is possible to improve the quality of the reintroduced fat.

### DISCLOSURE

The present application seeks to overcome the disadvantages of the prior art and concerns a machine for the purification of adipose tissue and introduction of the purified tissue into a patient's body, the machine comprising at least:
a material circulation circuit comprising at least (i) a purification device able to purify adipose tissue removed from the patient's body, the purification device comprising means for retaining adipose tissue and filtering contaminants delimiting a treatment volume intended to receive the adipose tissue, and a rotational drive means able to rotate the retaining and filtering means, and (ii) an introduction device able to communicate with the purification device for introducing purified adipose tissue into the patient's body, the circuit being further provided with means for controlling the circulation of material able to allow or prevent communication between elements of the circuit, and
a control unit configured to actuate the purification device and the control means, the control unit being configured to place the circuit in an adipose tissue purification configuration in which the retaining and filtering means are rotated to purify adipose tissue by centrifugation and filtration of contaminants, and the control unit being configured to place the circuit in a configuration for introducing purified adipose tissue into the patient's body in which purified adipose tissue is introduced from the purification device into the introduction device.

In a characteristic manner, the application implements a purification of adipose tissue by centrifugation and filtration of the contaminants through the retaining and filtering means due to the rotation of these means. This advantageously optimizes the quality of purified adipose tissue by reducing the presence of interstitial fluid and eliminating the presence of a blending or mixing member that could affect the integrity of adipose cells as in the case of the device marketed under the commercial reference Revolve® implemented in US 2020/0054824. The adipose tissue thus purified has, in particular, a much lower resorption rate than that obtained with the purification solutions of the prior art. In particular, the contaminants may be in the form of a liquid medium comprising at least one of oil, blood and water, possibly in a washing liquid. The application uses centrifugal assisted filtration in which the separation between adipose tissue and contaminants is facilitated by rotating the retaining and filtering means. The adipose tissue is subjected to centrifugal accelerations against the inner wall of the retaining and filtering means, allowing contaminants to be drained through the pores of these means while the adipose tissue is retained in the treatment volume. The solution according to the application also differs from centrifugation techniques in centrifuges where adipose tissue and contaminants are present in a completely closed container and which use very high accelerations, generally greater than 400 G, to achieve phase separation and can lead to damage to adipose tissue. Moreover, compared to these techniques, the purification according to the application makes it possible to eliminate the oil present, the reintroduction of which into the patient's body must be avoided because it can form oily cysts.

In an exemplary embodiment, the circuit further comprises: an inlet segment for the removal of adipose tissue from the patient's body that is connected to the purification device and a removal pump configured to introduce the adipose tissue removed from the inlet segment into the purification device, the circuit being able to define a closed circulation loop between the inlet segment and the introduction device with the patient's body, the control unit being configured to actuate the removal pump and place the circuit in a configuration for removing adipose tissue from the patient's body in which the removed adipose tissue is introduced into the purification device by the removal pump, and retained by the retaining and filtering means in the purification device.

Such a characteristic advantageously allows the machine to form a closed circuit with the patient that prevents the removed adipose tissue from being exposed to open air, thus avoiding any risk of contamination.

In particular, the inlet segment may be able to communicate with a suction inlet of the purification device and the circuit can include, in addition, a recovery tank connected to at least one suction outlet of the purification device and able to be placed into communication with the removal pump; the control unit can be configured to place the circuit in a purification configuration in which contaminants from the adipose tissue are suctioned by the removal pump through the at least one suction outlet of the purification device and collected in the recovery tank. Such a characteristic contributes to further increasing the compactness of the machine by using the same pump for the operations of removing adipose tissue and recovering contaminants.

In particular, the retaining and filtering means can be present between the treatment volume and a discharge volume distinct from the treatment volume, and the recovery tank can be connected to a first and a second suction outlet of the purification device, the first suction outlet opening into the treatment volume and the second suction outlet opening into the discharge volume.

Such a configuration using several suction outlets in the device facilitates the steps of removing adipose tissue and discharging contaminants.

In particular, the recovery tank may be connected to the inlet segment by a bypass segment, placed in bypass with respect to the purification device, and able to be placed into communication with the removal pump,
the control unit being configured to place the circuit in a configuration for removing excess adipose tissue from the patient's body in which the removal pump suctions excess adipose tissue through the bypass segment to collect it in the recovery tank.

Such a characteristic advantageously allows the machine to perform an additional liposuction of the patient with removal of an excess of adipose tissue compared to the quantity necessary for reintroduction into the body. In this case, this excess is not intended to be recovered in the purification device but rather to be directed into the recovery tank after passing through the bypass segment.

In an exemplary embodiment, the circuit further comprises a storage container for containing a liquid for washing adipose tissue, and a circulation pump able to place the storage container in communication with the purification device, the control unit being configured to place the circuit in an adipose wash configuration in which wash liquid is introduced from the storage container into the purification device by the circulation pump.

Such a characteristic allows the washing liquid to be integrated directly into the machine circuit, improving compactness and ensuring that the washing step does not introduce external contaminants.

In an exemplary embodiment, the circuit further comprises an additional storage container intended to contain an anaesthetic, the circulation pump able to place the additional storage container in communication with an outlet segment intended to introduce material into the patient's body,
the control unit being configured to place the circuit in an anaesthetic configuration in which anaesthetic is introduced from the additional storage container into the outlet segment by the circulation pump.

Such a characteristic makes it possible to further functionalize the machine, which makes it possible to perform the anaesthesia prior to the removal, while maintaining a compact solution by using the circulation pump, also used for washing adipose tissue.

In an exemplary embodiment, the circulation pump is a reversible flow pump able to place the storage container in communication with the purification device along a first flow direction, and the additional storage container with the outlet segment along a second flow direction different from the first flow direction.

In one embodiment, the circulation pump is a peristaltic pump.

Such a characteristic advantageously makes it possible to maintain the flow rate of circulating material with great precision. Moreover, only the inside of the peristaltic pump tube is in contact with the pumped product, making it possible to avoid any risk of contamination or damage to the pump.

The application also relates to an assembly for the purification of adipose tissue and introduction of the purified tissue into a patient's body, comprising at least:
a machine such as described above, and
a surgical device able to introduce purified adipose tissue into the patient's body connected to the introduction device.

In an exemplary embodiment, the circuit further comprises the input segment as described above, and the assembly further comprises a surgical device able to remove adipose tissue connected to the input segment.

### BRIEF DESCRIPTION OF THE DRAWINGS

FIG. 1 is a flowchart showing a succession of steps that can be implemented by a machine according to the application.

FIG. 2 shows an example of a machine according to the application for which the circuit is in the anaesthesia configuration.

FIG. 3 shows the example of a machine according to the application for which the circuit is in the configuration for removing adipose tissue.

FIG. 4 shows the example of a machine according to the application for which the circuit is in a configuration for removing excess adipose tissue.

FIG. 5 shows the example of the machine according to the application for which the circuit is in the configuration for washing adipose tissue.

FIG. 6 shows the example of a machine according to the application for which the circuit is in the configuration for suctioning contaminants.

FIG. 7 shows the example of a machine according to the application for which the circuit is in the configuration for introducing purified adipose tissue into the patient's body.

FIG. 8 shows a partial perspective view of an example of a machine according to the application.

FIG. 9 diagrammatically and partially shows a variant circulation pump that can be implemented within the scope of the application.

FIG. 10 shows a variant of the machine according to the application for which the introduction device is loaded without using the circulation pump.

FIG. 11 is an exploded schematic view of an example of a purification device that can be used in the context of the application.

FIG. 12 is a schematic sectional view of the purification device of FIG. 11 once assembled.

FIG. 13 is a schematic sectional view showing the purification device of FIG. 12 after displacement of the bottom.

### DESCRIPTION OF THE EMBODIMENTS

The following describes, with reference to the attached figures, an example of a machine 1 according to the application able to implement a treatment in several steps aimed at introducing purified adipose tissue into patient's body for an autologous adipose tissue transplantation procedure, for example, breast surgery or at other parts of the body. The following description describes these steps chronologically as well as the configurations of the machine 1 associated with each of them. In each figure, only the reference symbols of the elements relevant for the description of the action associated with the figure in question are indicated in order not to affect readability. In general, and as will be described in further detail below, the machine 1 comprises a material circulation circuit 2 which is controlled by a control unit 4 enabling the various treatment steps to be implemented. Before initiation of the treatment, the circuit 2 is in a sterile state.

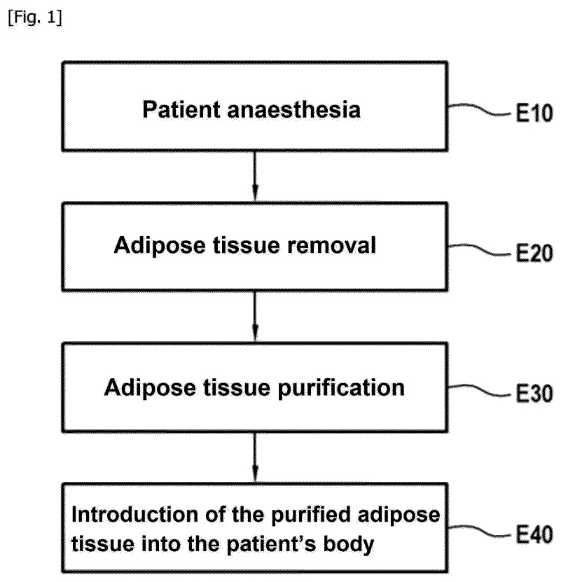

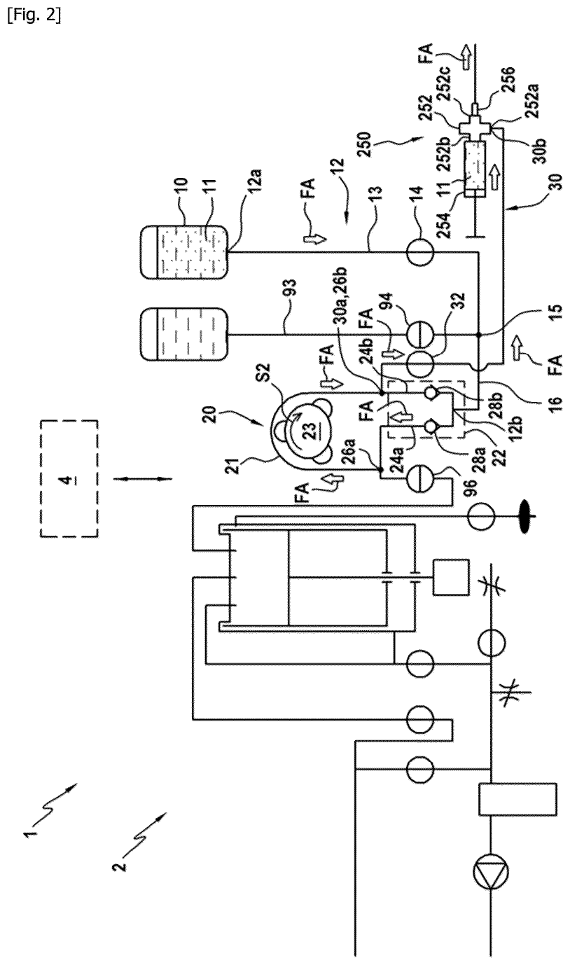

Anaesthesia (step E10 in FIG. 1) is performed prior to the removal of adipose tissue. The example of the machine 1 described with reference to FIGS. 2 to 7 is suitable for performing anaesthesia of the patient and the configuration of the circuit 2 corresponding to the anaesthesia step is illustrated in FIG. 2. The circuit 2 comprises a storage container 10 containing the anaesthetic 11 which is in liquid form, for example in the form of an aqueous solution, a circulation pump 20 and an outlet segment 30. The anaesthetic may be a local anaesthetic that allows local anaesthesia to be performed. The container 10 may be in the form of a flexible pouch, for example made of plastic. The segment 30 is intended for the introduction of material into the patient's body by being connected to an introduction device 250 able to allow this introduction. The device 250 comprises a body 252 provided with a first port 252a connected to the segment 30, a second port 252b connected to a syringe 254 and a third port 252c connected to an injection cannula 256. The first port 252a is provided with a check valve so as to allow the anaesthesia 11 to be injected into the patient through the cannula 256 by pressing the plunger of the syringe 254. The surgeon can act directly on the syringe 254 in order to induce the introduction of material into the patient's body, or in a variant, this action can be automated. The introduction device 250 is known in itself; for example, it is possible to use a device marketed under the reference LipoGrafter® by the company MTFBiologics.

The pump 20 here is a peristaltic pump, but variants are possible as described below. The circuit 2 comprises a segment 12 connecting the container 10 to an inlet 22 of the pump 20. The segment 12 has a first end 12a connected to the container 10 and a second end 12b, distinct from the first end 12a, connected to the inlet 22. The segment 12 here comprises two conduits 13 and 16 in the extension of each other. The conduit 16 extends the conduit 13 as far as the inlet 22 from a connecting node 15 at which the conduits 13, 16 and 93 intersect (the conduit 93 will be described below). The use of a common conduit 16 to extend the conduits 13 and 93 is advantageous for reducing the length of the tubing for the connection to the pump 20, thus simplifying the circuit 2. According to a variant not illustrated, the conduit 93 is not connected to the conduit 13 and is connected independently to the pump 20.

The inlet 22 defines two distinct supply conduits 24a, 24b each extending to a distinct orifice 26a, 26b of the body 21 of the pump 20. Each conduit 24a, 24b is provided with a check valve 28a, 28b. The body 21 of the pump 20 encloses the rotor 23 and the base of the pump 20 as well as the tubing extending between the orifices 26a and 26b through which the material is intended to flow. The conduit 24a, respectively 24b, extends between the segment 12 (or the conduit 16) and the orifice 26a, respectively 26b. A first end 30a of the segment 30 extends from the orifice 26b and a second end 30b of the segment 30 is connected to the introduction device 250.

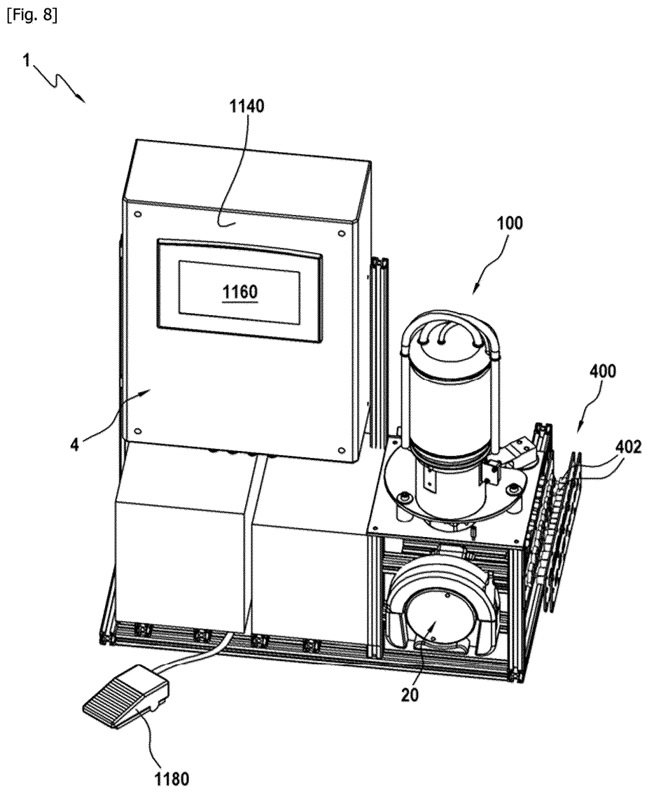

The segment 12 comprises a means 14 for controlling the circulation of material which is able to allow or prevent the transfer of material from the container 10 through this segment 12. In this case, the means 14 is present on the conduit 13 and enables communication between the container 10 and the pump 20 to be allowed or prevented. The segment 30 is also provided with a means 32 for controlling the circulation of material which allows or prevents the transfer of material from the pump 20 through the segment 30. In general, the segments and conduits used may be made of a flexible material, for example a polymer material, and the means for controlling the circulation of material may be capable of pinching the segments or conduits (“pinch valve”) to prevent the circulation of material and of releasing the pinch to allow this circulation. Such a solution is advantageous in order to simplify the design of the circuit, but the person skilled in the art will recognize that other solutions are possible for controlling the flow in the circuit. The control unit 4 acts on the means for controlling the circulation of material to allow or prevent communication between certain elements of the circuit as a function of the treatment step implemented by the machine 1. The control unit 4 generally comprises a control interface 1140 (see FIG. 8) on which the surgeon can especially select the step to be performed as a function of the progress of the operation. The control interface 1140 may comprise a screen 1160, touch-sensitive or otherwise, allowing the display of various information, such as information relating to the nature of the treatment step underway, the progress of this step, information relating to the patient, etc. The control interface 1140 also comprises at least one interaction element for controlling the machine 1 by the surgeon, comprising, for example, at least one of a set of keys or a pedal 1180. The surgeon chooses the step to be implemented by the machine on the control interface; this information is then transmitted to a control device of the control unit 4 which acts on the circuit 2, in particular on the pump 20 and the means for controlling the circulation of material, to put the circuit in the configuration associated with the selected treatment step. The surgeon can then initiate the desired treatment step by acting on the control interface, for example by pressing the pedal 1180. The treatment step can be performed without interruption when the surgeon maintains pressure on the pedal, but it may also be desirable for the step to be performed discontinuously, with several interruptions, these interruptions being caused by temporary release of the pedal. According to a variant, the treatment step can be initiated by the surgeon pressing on the pedal, then the pressure on the pedal can be released during the performance of the step and then the step can be stopped by the surgeon pressing on the pedal again. The machine 1 also comprises an element 400 for positioning the tubing comprising a plurality of pistons 402 capable of pinching the segments or conduits (“pinch valve”) in order to prevent the circulation of material and to release the pinching in order to allow this circulation. The person skilled in the art will recognize that other solutions for pinching the tubes are conceivable.

In the case of the anaesthesia step E10, the control device acts on the circuit 2 so as to put it in the configuration illustrated in FIG. 2 and then anaesthesia can be initiated by the surgeon pressing the pedal. The pump 20 is a reversible flow pump in the example shown. According to this configuration, the control device places the means 14 and 32 in a configuration allowing the circulation of material (called the “open configuration”) and actuates the pump 20 so that it causes a flow of material along the direction of flow S2. The means 94 and 96, which will be described below, are in a configuration which does not allow the circulation of material (known as the “closed configuration”) during anaesthesia. Likewise, the pump 60 and the purification device 100, mentioned below, are not actuated during this step. During anaesthesia, the liquid 11 is suctioned by the pump 20 through the segment 12 to be discharged into the segment 30 and thus introduced into the patient's body. The liquid 11 flows along the direction of flow S2 in which it is introduced from the container 10 into the segment 30 and then into the device 250 by the pump 20. The liquid 11 here is suctioned by the pump 20 through the conduit 24a and the orifice 26a in order then to be discharged through the orifice 26b toward the segment 30. The arrows FA represent the flow of the liquid 11 in the circuit 2 during anaesthesia as well as its injection into the patient through the cannula 256.

According to the example illustrated, the machine 1 is capable of performing anaesthesia, but it will be noted that if this is not the case, the anaesthesia being performed without using the machine, it will not exceed the scope of the application. In the latter case, the machine may, in accordance with a variant not illustrated, be devoid of container 10, segment 12 and conduit 24a. The rest of the treatment consisting of removing the adipose tissue from the patient's body will now be described (step E20). The adipose tissue thus removed is intended to be purified for subsequent reintroduction into the patient's body.

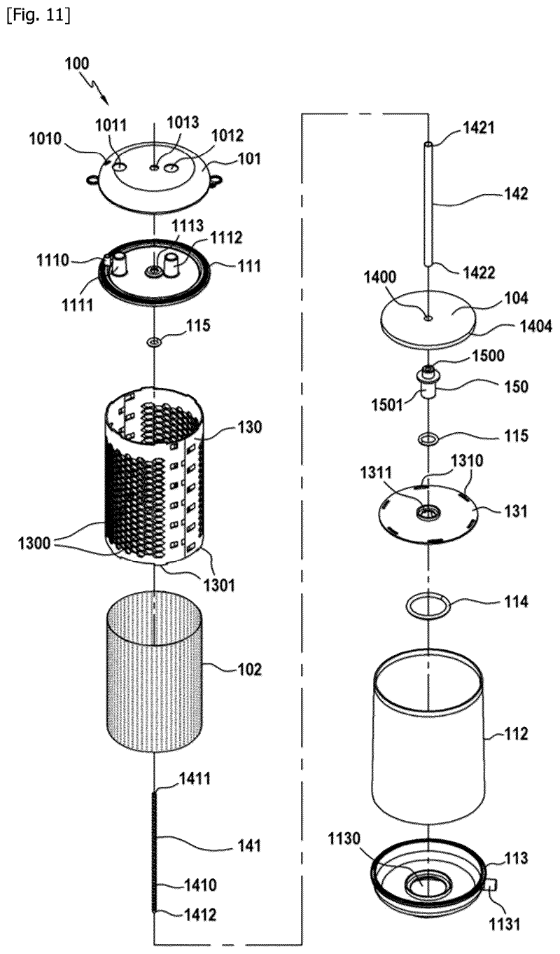

The circuit 2 comprises an inlet segment 40 intended to remove adipose tissue from the patient's body by being connected to a surgical device (not shown) capable of allowing this removal, such as a suction cannula. The segment 40 connects the surgical removal device to a purification device 100 which is capable of purifying the adipose tissue removed. An example of a device 100 especially comprising means 102 for retaining adipose tissue and for filtering contaminants will be described in greater detail hereinafter, with reference to FIGS. 11 to 13. The means 102 may be in the form of a filtering side wall. These means 102 have pores with a pore size configured to allow contaminants to pass through, for example in the form of a liquid medium, and retain adipose tissue. The segment 40 here comprises two conduits 42 and 44 in the extension of each other. The conduit 42 connects the surgical removal device to the connection node 45 at which the conduits 42, 44 and 80 intersect (the conduit 80 will be described below). The conduit 44 extends between a first end 44a at the node 45 and a second end 44b which opens into the device 100. The second end 44b, and therefore the segment 40, opens into a treatment volume V of the device 100 delimited by the means 102 and by a bottom 104. The segment 40 comprises a means 46 for controlling the circulation of material which is capable of allowing or preventing the transfer of material from the surgical removal device through the segment 40 to the device 100. In this case, the means 46 is present on the conduit 44. The circuit 2 also comprises a segment 50 which connects the device 100 to a removal pump 60, distinct from the pump 20 mentioned above. The pump 60 is a suction pump. The pump 60 can be placed in communication with the surgical removal device through the segment 50, the device 100 and the segment 40 to suction the adipose tissue from the patient's body. The pump 60 may be a vacuum pump. The segment 50 here comprises several ends 50a and 50b which open into the interior of the device 100 in order to effect a suction intake by the removal pump 60 in order to create a vacuum in the device 100; this allows the adipose tissue to be suctioned from the patient's body into the device 100 and then the contaminants of the adipose tissue present in the device 100 to be discharged. According to the example illustrated, a first end 50a of the segment 50 opens out into the treatment volume V and a second end 50b of the segment 50 opens out into a discharge volume VE of the device 100, distinct from the treatment volume V. The end 44b defines a suction inlet of the device 100 through which the adipose tissue removed is intended to be introduced into the device 100. The ends 50a and 50b define a suction outlet of the device 100, in communication with the pump 60, through which the contaminants of the adipose tissue are intended to be suctioned by the pump 60. The means 102 may be situated between the volumes V and VE. The volume VE may be situated around the means 102 and the volume V, as illustrated. An example with two ends 50a and 50b has been shown, but it would not exceed the scope of the application if more ends are present or if only one end is present, as long as the vacuum generated is sufficient to allow the suction of the adipose tissue and the discharge of the contaminants.

The segment 50 here comprises a connection node 55 at which the conduits 52, 54 and 58 intersect. The conduit 58 extends the conduits 52 and 54 beyond the node 55 and comprises a means 56 for controlling the circulation of material able to place the pump 60 in communication with the device 100 or of preventing this communication. The segment 50, in particular the conduit 58, is connected to vacuum regulators 62 and 64 which make it possible to maintain a predefined vacuum level during suction by the pump 60. According to the example illustrated, a solenoid valve 66 is present between the vacuum regulator 64 and the segment 50. The circuit 2 also comprises a conduit 70 having a first end 70a opening into the device 100 (in the volume VE in the example illustrated) and a second end 70b opening into the open air and connected to an air filter 74. The communication through the conduit 70 is controlled by the means 72 for controlling the circulation of material.

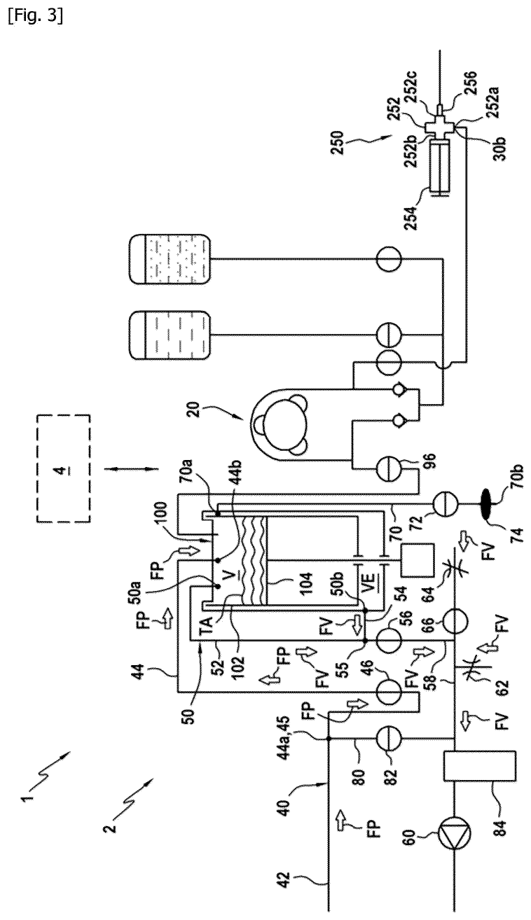

Step E20 of adipose tissue removal can be initiated by the action of the surgeon on the control interface as described above. The control device acts on the circuit 2 so as to place it in the configuration illustrated in FIG. 3 in which the means 46 and 56 are in the open configuration, as is the solenoid valve 66, and the pump 60 is actuated. The means 82 and 96, which will be described hereinafter, and the means 72 are in closed configuration during this step. Similarly, the pump 20 is not actuated. During removal, the adipose tissue is suctioned from the patient's body through the segment 40, by the action of the pump 60, in order to be introduced into the device 100 through the end 44b. The adipose tissue TA removed is then retained in the device 100 by the means 102. The arrows FP represent the transfer of the adipose tissue TA removed in the circuit 2. The arrows FV represent the drawing of the vacuum produced by the pump 60 downstream of the device 100 in order to suction the adipose tissue TA.

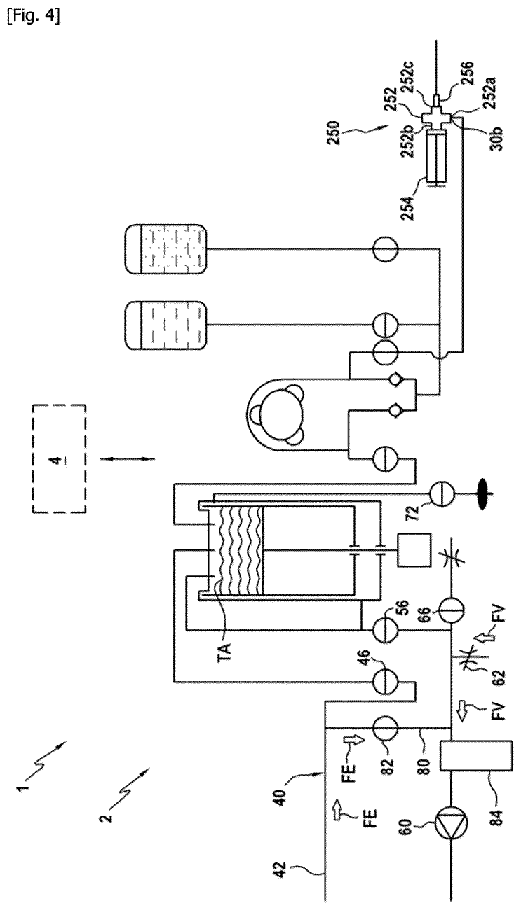

The removed adipose tissue TA present in the device 100 is then washed. Note, however, FIG. 4 which illustrates a variant in which removal is continued in the case where the surgeon wishes to carry out an additional liposuction with removal of an excess of adipose tissue compared to the quantity necessary for reintroduction into the body. In this case, this excess is not intended to pass into the device 100 but rather to be recovered in a recovery tank 84 in communication with the pump 60. The tank 84 constitutes a waste container and is intended to recover elements which are not intended for introduction into the patient. In the case illustrated in FIG. 4, the control device places the means 46 and 56 and the solenoid valve 66 in the closed configuration and opens the means 82 for controlling the circulation of material when sufficient adipose tissue TA has been taken from the device 100. The pump 60 is still actuated. The means 82 is present on a bypass segment 80 which is placed in bypass with respect to the device 100 and is capable of being put into communication with the pump 60. The segment 80 extends between the node 45 and the tank 84 and the excess adipose tissue is suctioned by the pump 60 through the conduit 42 and the segment 80 to be recovered in the tank 84 without passing through the device 100 (arrows FE in FIG. 4).

According to the example illustrated, the machine 1 is capable of removing the adipose tissue. This aspect is advantageous because it allows the circuit 2 to define a closed circulation loop between the segment 40 and the device 250 with the patient's body. Nevertheless, according to a non-preferential variant of the application but covered by the wording of the claims, the conduits 42, 44 and 80 may be omitted and the filling of the device 100 with the removed adipose tissue may be carried out using a third-party device not forming an integral part of the machine in view of the purification of the tissue for reintroduction into the body. The structure of an example of a device 100 allowing purification of the adipose tissue removed will now be described below in relation to FIGS. 11 to 13. It will be noted that, in the case where an excess of adipose tissue is removed, at least part of the purification can be carried out at the same time as the removal of this excess, in order to reduce the treatment time.

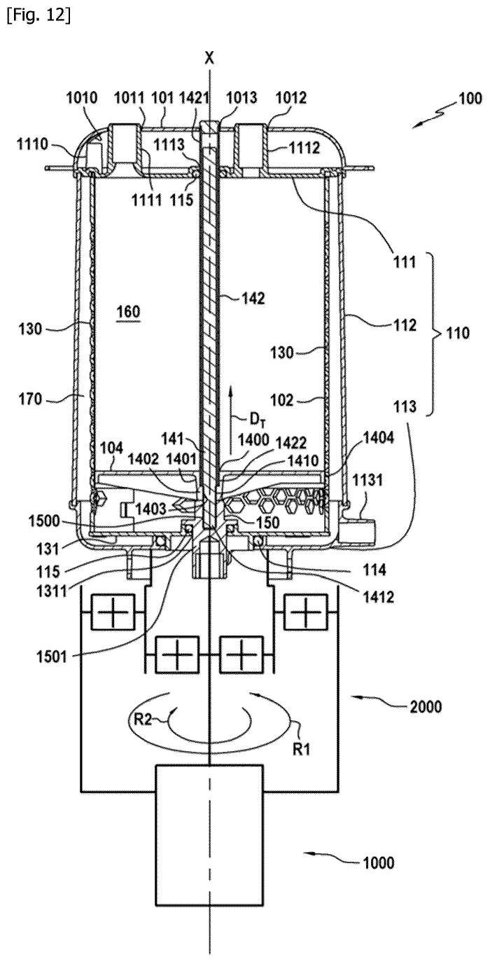

The purification device 100 comprises a sealed enclosure 110 formed here by a cap 111, a side wall 112 and a bottom 113. These elements are secured together in a sealed manner. The means 102 are present in the sealed enclosure 110. The means 102 delimit a centrifugation chamber 160 (visible in FIG. 12 and corresponding to volume V described previously). In the example described here, the means 102 have a cylindrical shape, but the person skilled in the art will recognize that the means 102 can have other shapes suitable for centrifugation. As indicated above, the means 102 have a pore size configured to allow a liquid medium to pass through and retain adipose tissue. The pore size is chosen, in particular, to allow the passage of liquids such as oil, blood or physiological solution while retaining adipose tissue. In general, the pore size of the means 102 can be less than or equal to 1.5 mm, for example less than or equal to 0.5 mm. This size can be comprised between 0.05 mm and 1.5 mm, for example, between 0.2 mm and 0.5 mm. The means 102 can be made of a polymer material, for example polyester or polypropylene, but the person skilled in the art will recognize that other materials can be used.

In the example described here, a stiffening element 130 is present between the wall 112 of the sealed enclosure 110 and the means 102. Its function is especially to ensure the structural strength of the means 102 during centrifugation. The stiffening element 130 is, for example, made of a metal or plastic material and has a perforated structure defining a plurality of openings 1300 in order to allow the discharge of the liquid medium drained by the means 102.

The stiffening element 130 is associated with a rotary plate 131. More precisely, the stiffening element 130 comprises at its lower end teeth 1301 which cooperate with grooves 1310 present in the vicinity of the outer periphery of the rotary plate 131. The person skilled in the art will recognize that the element 130 can be connected to the plate 131 in different ways. The rotary plate 131 is connected to a rotary drive means which can be manual or motorized. In the example described here, the rotary plate is connected to an electric motor 1000, such as a stepper motor or a brushless DC motor, via a two-way clutch 2000 configured to drive the rotary plate 131 in rotation in a first direction of rotation R1 (FIG. 12).

Gaskets 114 and 115 are placed respectively below and above the rotary plate 131 in order to ensure sealing in the lower part of the purification device.

Centrifugation is carried out by rotating the means 102. More precisely, the electric motor 1000 is controlled in the first direction of rotation R1 to drive the rotary plate 131 and the stiffening element 130 into engagement with the plate 131. The rotation of the plate 131 and of the stiffening element 130 causes the rotation of the means 102 which are of one piece with the stiffening element 130, for example by being glued or clipped to this element. The speed of the electric motor 1000 is controlled so as to apply a centrifugal force to the material in the centrifugation chamber 160. Thus, an adipose tissue present in the centrifugation chamber 160 will be subjected to a centrifugal force against the internal wall of the means 102, which makes it possible to efficiently drain the liquid medium present in the adipose tissue without damaging this tissue.

During centrifugation, the liquid medium passing through the means 102 and the stiffening element 130 is collected in a volume 170 (corresponding to the discharge volume VE described previously) delimited between the stiffening element 130 and the wall 112 of the enclosure 110. The liquid is then discharged via a discharge port 1131 present on the bottom 113 of the enclosure 110, the port 1131 defining the orifice 50b described above. In the example described here, the cap 111 comprises three ports 1110, 1111 and 1112 intended to be connected, respectively, to a suction device for adipose tissue taken from the patient's body (conduit 52 described above), the conduit 44 for the introduction of the adipose tissue removed and a device for delivering the washing liquid (conduit 95 described below). The purification device comprises a cover 101 comprising openings 1010, 1011 and 1012 which cooperate with the ports 1110, 1111 and 1112 of the cap 111.

The purification device can also comprise a bottom 104 of the centrifugation chamber 160, for example in the form of a collecting tray, which is movable inside the centrifugation chamber. As illustrated in the example of FIGS. 11 and 12, the purification device 100 further comprises a collecting tray 104 movable in translation in a direction DT along the axis X of the means 102. More precisely, the purification device 100 comprises a threaded shaft 141 which extends vertically inside the centrifugation chamber 160 along the axis X of the means 102. The lower end 1412 of the threaded shaft is connected to the electric motor 1000 by means of a guide 150 and the two-way clutch 2000. The guide 150 comprises a housing 1500 in which the lower end 1412 is secured. A lower portion 1501 of the guide 150 is connected to a part of the two-way clutch 2000 which is engaged with the electric motor 1000 only when this motor transmits a rotational movement along a second direction of rotation R2 opposite to the first direction of rotation R1 used for the centrifugation. When the electric motor 10 rotates in the first direction of rotation R1, no rotational movement is transmitted by the two-way clutch 2000 to the guide 150 to which the threaded shaft 141 is connected. When the motor 1000 rotates in the second direction of rotation R2, no rotational movement is transmitted by the two-way clutch 2000 to the rotary plate 131 and, consequently, to the means 102. The collecting tray 104 comprises, on its upper face, a central opening 1400 extended by a neck 1401 which extends from the lower face of the tray 104. The neck 1401 comprises a portion 1402 comprising a thread 1403 which cooperates with a thread 1411 of the threaded shaft 141. Thus, when the threaded shaft is driven in rotation in the second direction of rotation R2, the collecting tray 104 rises in the centrifugation chamber 160 in the direction DT.

The peripheral edge 1404 of the collecting tray 104 is opposite the internal wall of the means 102. Thus, when the collecting tray 104 is moved in vertical translation along the direction DT, this acts as a piston that scrapes the inner wall of the filter to collect more adipose tissue and facilitate the discharge of purified adipose tissue from the top of the device for introduction into the patient's body via the device 250. According to a particular aspect, the threaded shaft 141 can be housed in a protective sheath 142. The protective sheath 142 which extends between an upper end 1421 and a lower end 1422 makes it possible to prevent the threaded shaft 141 from coming into contact with the adipose tissue which, by accumulating at thread 1403 of the collecting tray 104, can block the displacement of this tray.

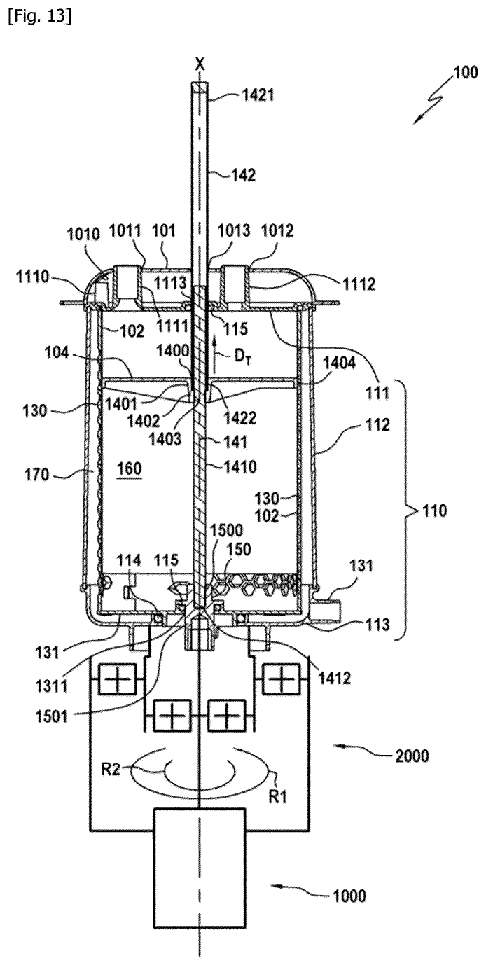

FIG. 13 shows the purification device 100 after actuation of the electric motor 1000 in the second direction of rotation R2 enabling the threaded shaft 141 to be driven in rotation. The rotation of the threaded shaft 141 causes the collecting tray 104 to move vertically in translation in the direction DT.

The lower end 1422 of the protective sheath is secured in the opening 1400 of the collecting tray 104. In order to allow the movement of the protective sheath 142 during the displacement of the tray 104, the cap 111 and the cover 101, respectively, comprise an opening 1113 and an opening 1013 through which the sheath 142 slides. A gasket 115 is present around the opening 1113 in order to preserve the seal at the top of the centrifugation chamber.

In another exemplary embodiment, the means 102 are made of a rigid self-supporting material such as, for example, a metal material. In this case, the stiffening element 130 is no longer necessary and it is the self-supporting means 102 which are directly engaged with the rotary plate 131.

According to a variant not illustrated, it will be noted that it is possible to add a turbine circulation system making it possible to improve the circulation of liquids in the device 100, in particular of the washing liquid.

In general, the device 100 allows purification by centrifugation of the adipose tissue TA removed. It should be noted that several centrifugation phases can be carried out at different times of the method and in particular while the adipose TA tissue is removed, or during the introduction of the washing liquid in order to stir all the materials present in the volume V and avoid any risk of clogging of the means 102 by the adipose tissue.

In general, a centrifugation phase is carried out by rotating the means 102. The means 102 are rotated around themselves. The means 102 are rotated around the axis X (see FIG. 12). The axis X can correspond to the axis of the height of the means 102, and for example, to an axis of symmetry or revolution of these means 102. During this rotation, the adipose tissue and the contaminants can undergo an acceleration greater than or equal to 8 G, for example greater than or equal to 10 G or greater than or equal to 12 G. This acceleration measured in G corresponds to the ratio between the acceleration undergone by the material and the acceleration of Earth's gravity, which is approximately 9.81 m²/s. The acceleration experienced by the material corresponds to the ratio of the centrifugal force applied to the mass of the material concerned. The centrifugal force applied is equal to m*ω²*R where m is the mass of the object in question, w is the angular velocity of the means 102 expressed in rad/s and R is the distance from the axis X of rotation to the centre of gravity of the object in question. During a centrifugation phase, the adipose tissue and the contaminants can undergo an acceleration less than or equal to 40 G, for example less than or equal to 30 G, or less than or equal to 25 G or less than or equal to 20 G. This acceleration can be comprised between 8 G and 40 G or between 8 G and 30 G or between 8 G and 25 G or between 8 G and 20 G. This acceleration can be comprised between 10 G and 40 G or between 10 G and 30 G or between 10 G and 25 G or between 10 G and 20 G. This acceleration can be comprised between 12 G and 40 G or between 12 G and 30 G or between 12 G and 25 G or between 12 G and 20 G. The acceleration values described above make it possible to optimize the elimination of fluids present in the adipose tissue and especially interstitial fluid and further improve the quality of the purified adipose tissue. This last point is more difficult to achieve in the case of gravity filtration as described in US 2020/0054824. Moreover, limiting acceleration to values much lower than those used in conventional centrifugation as described above, also contributes to obtaining adipose tissue of optimal quality and with a small quantity of residual interstitial fluid allowing it to be easily injected into the patient's body.

The duration of a centrifugation cycle can typically be greater than or equal to 10 seconds. This duration can be less than or equal to 60 seconds. This duration can, for example, be comprised between 10 seconds to 60 seconds, for example between 15 seconds to 45 seconds. It should be noted that it is possible to carry out several centrifugation cycles with stopping the rotation of these means between these cycles at different steps of the method, for example an alternation of centrifugation cycles with stopping the rotation during the removal of the adipose tissue or during washing.

Purification of the adipose tissue removed (step E30) is initiated by the surgeon as described above and can therefore be initiated during the removal of the adipose tissue. Purification especially comprises washing the adipose tissue which will now be described.

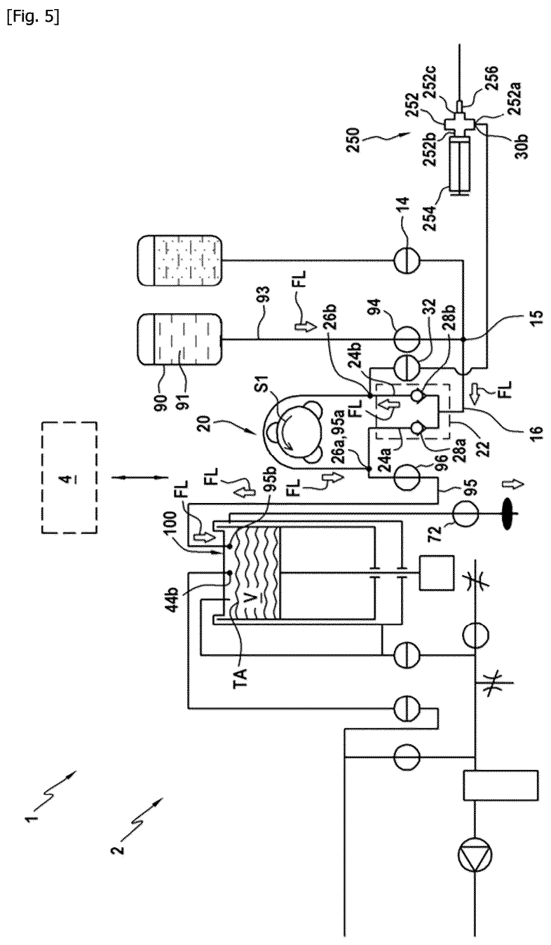

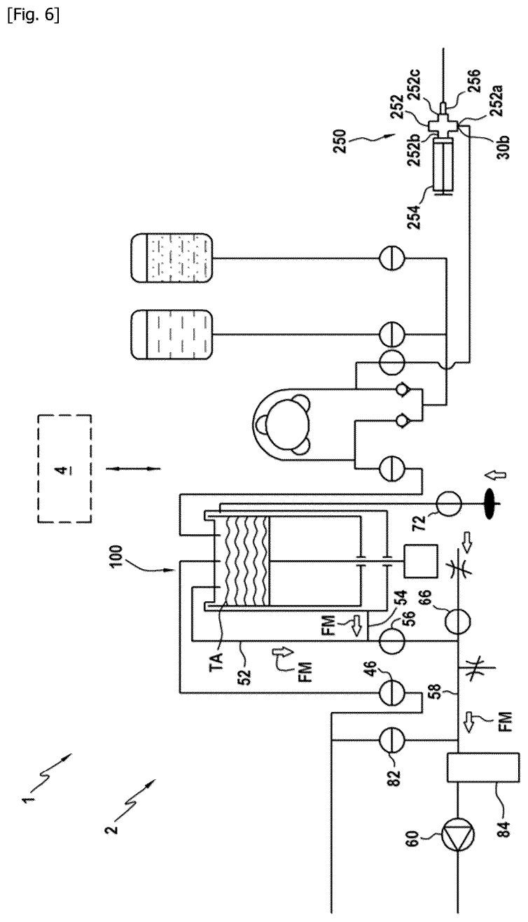

FIG. 5 illustrates the configuration of the circuit 2 allowing the washing of the adipose tissue. The circuit 2 comprises a container 90 containing the washing liquid 91. The liquid 91 can be a physiological solution. The container 90 can be in the form of a flexible pouch, for example made of plastic. The container 90 is connected to the inlet 22 of the pump 20 by means of the conduits 93 and 16 in the example illustrated. The conduit 93 comprises a means 94 for controlling the circulation of material which is capable of allowing or preventing the transfer of material from the container 90 through this conduit 93. The circuit 2 also comprises a conduit 95 connecting the pump 20 to the device 100. The conduit 95 here extends between a first end 95a at the orifice 26a and a second end 95b which opens into the volume V of the device 100. The conduit 95 is distinct from the conduit 24a which also extends as far as the orifice 26a. The end 95b may be distinct from the end 44b described previously or, according to a variant not illustrated, the conduits 95 and 44 may be joined together in a single conduit opening into the volume V of the device 100. In the example illustrated, the device 100 is connected to an orifice 26a of the body 21 of the pump 20 which is distinct from the orifice 26b to which the segment 30 is connected. The conduit 95 comprises a means 96 for controlling the circulation of material which is capable of placing the pump 20 in communication or not with the device 100. According to the washing configuration of FIG. 5, the means 94, 96 and 72 are in the open configuration and the means 14 and 32 are in the closed configuration. The pump 20 is actuated and causes a flow of material along the direction of flow S1, opposite to the direction of flow S2 described above. The liquid 91 is suctioned from the container 90 by the pump 20 to be discharged into the conduit 95 and thus introduced into the device 100. The liquid 91 flows along the direction of flow S1 in which it crosses the conduits 93 and 16 up to the inlet 22 of the pump 20. The liquid 91 is suctioned by the pump 20 through the conduit 24b and the orifice 26b in order then to be discharged through the orifice 26a and the conduit 95 to the device 100. In FIG. 5, the arrows FL show the flow of the liquid 91 in the circuit 2 during washing. During the introduction of the washing liquid into volume V, one or more centrifugation cycles are carried out as described above to separate the contaminants. Suction by the pump 60 is then carried out in order to discharge the washing liquid containing the contaminants (water, blood, oil, etc.) from the adipose tissue and recover it in the tank 84. FIG. 6 illustrates the configuration of the circuit 2 allowing this suction. The means 82 are in the closed configuration and the means 56 and 72 are in the open configuration, as is the solenoid valve 66. The pump 60 is actuated. In the example illustrated, the contaminants (arrows FM) are suctioned out of the device 100 through the conduits 52, 54 and 58 in order to be recovered in the tank 84.

After this suction, a centrifugation cycle can be carried out again, if desired, to remove more contaminants. This washing/suction/centrifugation sequence can then be repeated if necessary.

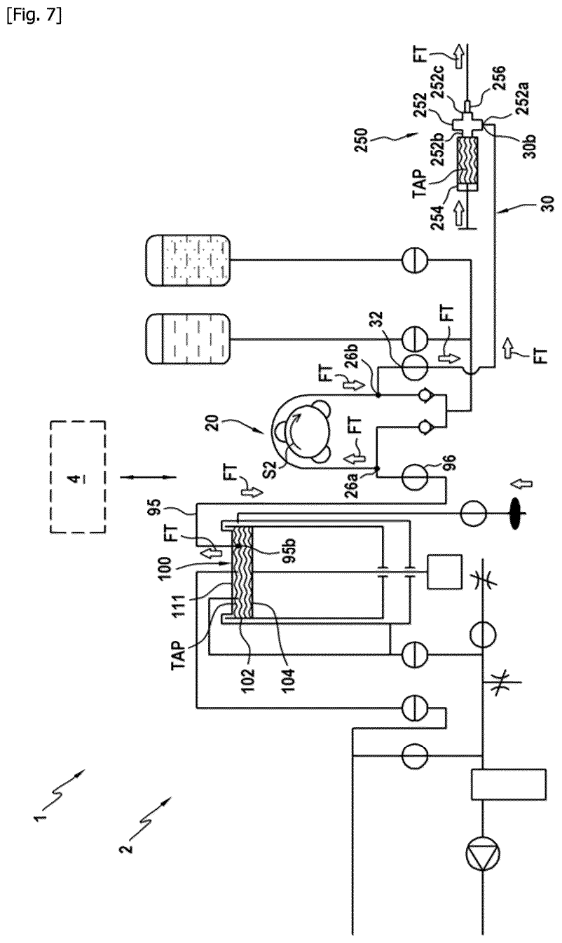

If desired, purification can be completed after the sequences described above by compacting the adipose tissue in which the bottom 104 can be raised, such as a piston (see position of the bottom 104 in FIGS. 7 and 13), and suction can be carried out on the compacted adipose tissue, similar to what has just been described, to discharge the air and any contaminants that may still be present.

Once purification is complete, the surgeon activates the step of reintroduction into the patient's body (step E40 in FIG. 1). In the example illustrated in FIG. 7, the purified adipose tissue TAP is introduced into the segment 30 by the pump 20 along the flow direction S2, opposite to that used for introducing the liquid 91 into the device 100. In this example, a slight pressure is applied to the purified adipose tissue in order to press it against the cap 111 of the device 100 due to the rise of the bottom 104 and proceed to discharge it toward the segment 30 through the end 95b. The pump 20 suctions the purified adipose tissue through the conduit 95, from the orifice 26a and the purified adipose tissue TAP is then discharged through the orifice 26b to be introduced into the segment 30 for introduction into the device 250 which will allow the purified tissue TAP to be reintroduced into the patient. The arrows FT in FIG. 7 represent the transfer of purified adipose tissue and its reintroduction into the patient by the device 250.

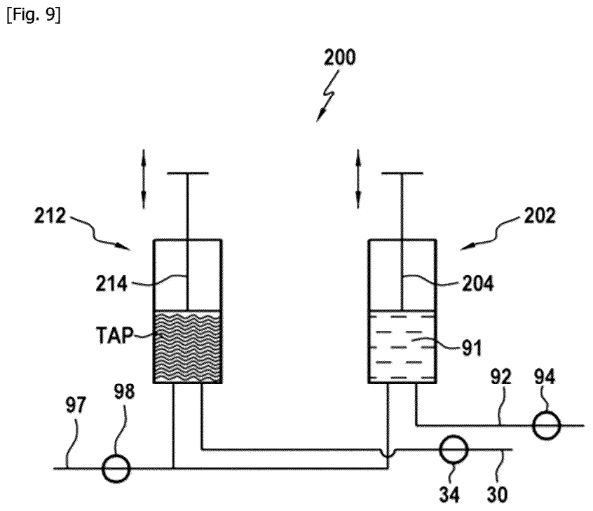

The description which has just been given, with reference to FIGS. 2 to 8, concerns an example where the circulation pump is a peristaltic pump. However, the application is not limited to such a solution. A variant is illustrated in FIG. 9, in which the circulation pump 200 is formed by a multi-syringe system. According to this variant, the pump 200 comprises at least one first syringe 202 with a plunger 204 which is configured to suction the washing liquid 91 in particular through the conduit 92 in order to then inject it into the device 100 by transfer into the conduit 97 in order to carry out the washing of the adipose tissue described above. The pump 200 comprises a second syringe 212 with a plunger 214 which is configured to suction the purified adipose tissue TAP from the device 100 through the conduit 97 in order to then inject it into the outlet conduit 30 and then into the device 250 for reintroduction into the patient's body. A case has been shown in which the conduits 92 and 97 have distinct entry points into the syringe 202 (the same applies to the conduits 97 and 30 in the syringe 212); however, it does not exceed the scope of the application when these conduits open into the syringe through a common orifice. FIG. 9 shows the case of a multi-syringe system, but a circulation pump consisting of a single syringe could also be used.

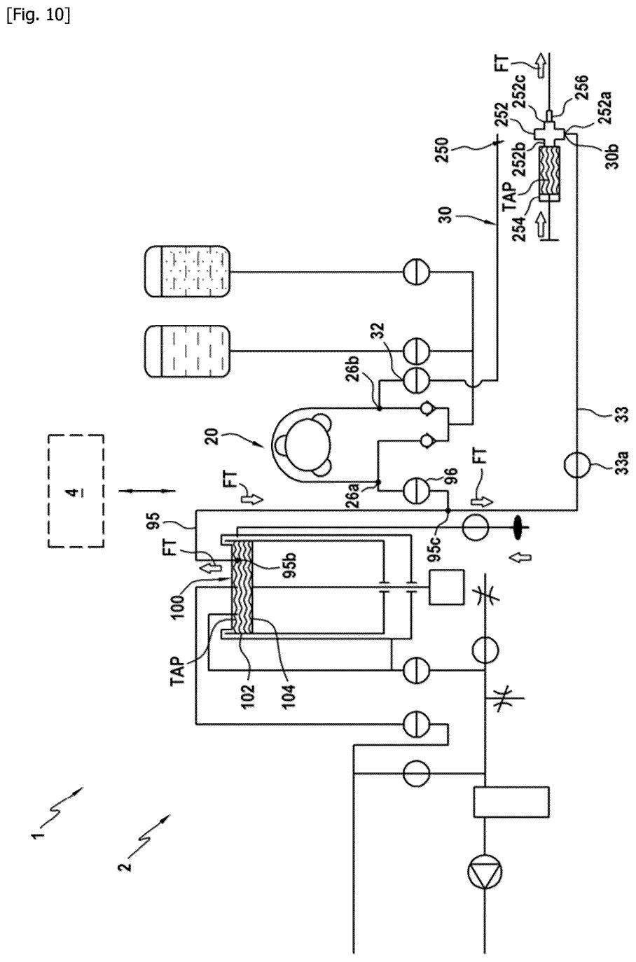

According to another variant described in FIG. 10, the introduction of the purified adipose tissue TAP into the device 250 is not carried out using the pump 20. In fact, the conduit 95 has a node 95c from which extends a conduit 33 distinct from the conduit 30 and provided with a flow control means 33a, here in the open position. The conduit 33 connects the conduit 95 to the device 250 for reintroducing the purified tissue TAP into the patient's body. Therefore, it is not necessary to use the pump 20 for the reintroduction of purified adipose tissue TAP into the patient's body.

The expression “comprised between . . . and . . . ” should be understood to include the bounds.

1. A machine for the purification of adipose tissue and introduction of the purified tissue into a patient's body, the machine comprising:
a material circulation circuit comprising at least (i) a purification device able to purify adipose tissue removed from the patient's body, the purification device comprising means for retaining adipose tissue and filtering contaminants delimiting a treatment volume intended to receive the adipose tissue, and a rotational drive means able to rotate the retaining and filtering means, and (ii) an introduction device able to communicate with the purification device for introducing purified adipose tissue into the patient's body, the circuit being further provided with means for controlling the circulation of material able to allow or prevent communication between elements of the circuit, and
a control unit configured to actuate the purification device and the control means, the control unit being configured to place the circuit in an adipose tissue purification configuration in which the retaining and filtering means are rotated to purify adipose tissue by centrifugation and filtration of contaminants, and the control unit being configured to place the circuit in a configuration for introducing purified adipose tissue into the patient's body in which purified adipose tissue is introduced from the purification device into the introduction device,
wherein the circuit further comprises an inlet segment intended to collect adipose tissue from the patient's body that is connected to the purification device and a removal pump configured to introduce adipose tissue removed from the inlet segment into the purification device, the circuit being able to define a closed circulation loop between the inlet segment and the introduction device with the patient's body;
the control unit being configured to actuate the removal pump and place the circuit in a configuration for removing adipose tissue from the patient's body in which the removed adipose tissue is introduced into the purification device by the removal pump, and retained by the retaining and filtering means in the purification device, wherein the inlet segment is able to communicate with a suction inlet of the purification device and the circuit further comprises a recovery tank connected to at least one suction outlet of the purification device and able to be placed into communication with the removal pump;
the control unit being configured to place the circuit in a purification configuration in which adipose tissue contaminants are suctioned by the removal pump through the at least one suction outlet of the purification device and collected in the recovery tank;
wherein the recovery tank is connected to the inlet segment by a bypass segment, placed in bypass with respect to the purification device, and able to be placed into communication with the removal pump;
the control unit is configured to place the circuit in a configuration for collecting excess adipose tissue from the patient's body, wherein the removal pump suctions excess adipose tissue through the bypass segment to recover it into the recovery tank.

2. (canceled)

3. (canceled)

4. The machine according to claim 13, wherein the retaining and filtering means are present between the treatment volume and a discharge volume distinct from the treatment volume, and wherein the recovery tank can be connected to a first and a second suction outlet of the purification device, the first suction outlet opening into the treatment volume and the second suction outlet opening into the discharge volume.

5. (canceled)

6. The machine according to claim 1, wherein the circuit further comprises a storage container for containing a liquid for washing the adipose tissue, and a circulation pump able to place the storage container in communication with the purification device, the control unit being configured to place the circuit in an adipose tissue wash configuration in which wash liquid is introduced from the storage container into the purification device by the circulation pump.

7. The machine of claim 6, wherein the circuit further comprises an additional storage container for containing an anaesthetic, the circulation pump being able to place the additional storage container in communication with an outlet segment for introducing material into the patient's body,
the control unit being configured to place the circuit in an anaesthetic configuration in which anaesthetic is introduced from the additional storage container into the outlet segment by the circulation pump.

8. The machine according to claim 7, wherein the circulation pump is a reversible flow pump able to place the storage container in communication with the purification device along a first flow direction, and the additional storage container with the outlet segment along a second flow direction (S2) different from the first flow direction.

9. The machine according to claim 6, wherein the circulation pump is a peristaltic pump.

10. An assembly for the purification of adipose tissue and introduction of the purified tissue into a patient's body, comprising at least:
a machine according to claim 1, and
a surgical device able to introduce purified adipose tissue into the patient's body connected to the introduction device.

11. The assembly according to claim 10, wherein the assembly further comprises a surgical device able to remove adipose tissue connected to the inlet segment.

12. A machine for purification of adipose tissue and introduction of the purified tissue into a patient's body, the machine comprising:
a material circulation circuit comprising at least (i) a purification device to purify adipose tissue removed from the patient's body, and (ii) an introduction device for introducing purified adipose tissue into the patient's body;
a control unit configured to actuate the purification device, the control unit being configured to place the circuit in an adipose tissue purification configuration in which to purify adipose tissue by centrifugation and filtration of contaminants, the control unit being configured to place the circuit in a configuration for introducing purified adipose tissue into the patient's body in which purified adipose tissue is introduced from the purification device into the introduction device;
wherein the circuit further comprises an inlet segment intended to collect adipose tissue from the patient's body that is connected to the purification device and a removal pump configured to introduce adipose tissue removed from the inlet segment into the purification device, the circuit being able to define a closed circulation loop between the inlet segment and the introduction device with the patient's body;
the control unit being configured to actuate the removal pump and place the circuit in a configuration for removing adipose tissue from the patient's body in which the removed adipose tissue is introduced into the purification device by the removal pump, and retained in the purification device, wherein the inlet segment is able to communicate with a suction inlet of the purification device and the circuit further comprises a recovery tank connected to at least one suction outlet of the purification device and able to be placed into communication with the removal pump;
the control unit being configured to place the circuit in a purification configuration in which adipose tissue contaminants are suctioned by the removal pump through the at least one suction outlet of the purification device and collected in the recovery tank;
wherein the recovery tank is connected to the inlet segment by a bypass segment, placed in bypass with respect to the purification device, and able to be placed into communication with the removal pump; and
the control unit is configured to place the circuit in a configuration for collecting excess adipose tissue from the patient's body, wherein the removal pump suctions excess adipose tissue through the bypass segment to recover it into the recovery tank.

13. A method for purifying adipose tissue, the method comprising:
actuating a removal pump and removing adipose tissue from a patient's body;
introducing the removed adipose tissue into a purification device by way of the removal pump;
suctioning adipose tissue contaminants and collecting the adipose tissue contaminants in a recovery tank; and
collecting excess adipose tissue from the patient's body, wherein the removal pump suctions excess adipose tissue through a bypass segment to recover it into the recovery tank.
